(19) United States
(12) Patent Application Publication
(10) Pub. No.: US 2015/0018822 A1
(43) Pub. Date: Jan. 15, 2015

(54) NEEDLE ASSEMBLIES AND SYSTEMS FOR USE IN ABLATION PROCEDURES AND RELATED METHODS

(71) Applicant: N. Sandor Racz, Coppell, TX (US)

(72) Inventor: N. Sandor Racz, Coppell, TX (US)

(73) Assignee: Custom Medical Applications, Inc., Johnstown, NY (US)

(21) Appl. No.: 14/376,778

(22) PCT Filed: Feb. 8, 2012
(86) PCT No.: PCT/US2012/024328

§ 371 Date: Aug. 5, 2014

(51) Int. Cl. A61B 18/14

(52) U.S. Cl. CPC A61B 18/1477; A61B 2018/00577
USPC 606/41; 29/825

(57) ABSTRACT

Needle assemblies for use in ablation procedures include a needle having an electrically conductive portion and at least one conductive member extending at least partially through a bore of the needle. A portion of the at least one conductive member is physically and electrically connected to the electrically conductive portion of the needle. Ablation systems and methods of ablation may include such needle assemblies. Methods of forming needle assemblies for use in ablation procedures include disposing at least one conductive member within a needle and physically and electrically connecting the at least one conductive member to an electrically conductive portion of the needle.

### CROSS-REFERENCE TO RELATED APPLICATIONS

This application is a national phase entry under 35 U.S.C. §371 of International Patent Application PCT/US2012/024328, filed Feb. 8, 2012, designating the United States of America and published in English as International Patent Publication WO2013/119224 A1 on Aug. 15, 2013.

### TECHNICAL FIELD

The disclosure relates generally to medical devices and associated methods. More specifically, disclosed embodiments relate to needle assemblies and systems for use in ablation procedures.

### BACKGROUND

High frequency ablation generally involves the removal or destruction of dysfunctional tissue (e.g., cancerous tissue, painful nervous tissue, or otherwise dysfunctional tissue) utilizing heat generated from high frequency, alternating current flowing to the dysfunctional tissue. Conventionally, current alternating at high frequencies, such as radio frequencies or microwave frequencies, is pulsed to an electrode (e.g., a radio frequency probe thermocouple) inserted into a subject. The alternating current flows from the electrode, through an ablation instrument (e.g., a needle) to which the electrode is connected, to the tissue to be removed. Tissue heat is generated by the flow of current through the electrical resistance offered by the tissue. The greater this resistance, the greater the heat generated. The current typically flows through the tissue to a grounding pad. Conventionally, current spreads out radially from the conductive ablation tip of the ablation instrument, so that current density is greatest next to the tip, and decreases progressively as distance from the tip increases. The frictional heat produced from ionic agitation is proportional to current (i.e., ionic density). Therefore, the heating effect is greatest next to the tip and decreases as distance from the tip increases.

For example, U.S. Patent Application Publication US 2009/0187179 A1, published Jul. 23, 2009, to Racz, the disclosure of which is incorporated herein in its entirety by this reference, discloses an ablation instrument. Briefly, an ablation instrument comprising a lesion wire extends from an interior lumen of a body, through infusion ports, to an exterior side of the body. The lesion wire is at least partially isolated from an opposing side of the body because of its protrusion from the body on the side on which the ports are located. Other energy emitting ablation elements are disclosed in, for example, U.S. Pat. No. 4,641,649, issued Feb. 10, 1987, to Walinsky et al., the disclosure of which is incorporated herein in its entirety by this reference, wherein a microwave ablation apparatus is disclosed.

A trend in the art has been to ensure the ablation procedure is complete and not overdone. A so-called “complete” ablation procedure commonly means that the ablation extends through the thickness of the tissue to be ablated before the application of ablation energy is stopped. U.S. Pat. No. 6,648,883, issued Nov. 18, 2003, to Francischelli et al., the disclosure of which is incorporated herein in its entirety by this reference, refers to this cut depth or ablation completion as “transmural” ablation. Briefly, a system and method for creating lesions and assessing their completeness or transmurality by monitoring the impedance of the tissue to be ablated is disclosed. An impedance measurement that is stable at a predetermined level for a certain time is monitored.

Other methods are disclosed in the art for detecting transmural ablation, such as, for example, detecting a desired drop in electrical impedance at the electrode site as in U.S. Pat. No. 5,562,721, issued Oct. 8, 1996, to Marchlinski et al., the disclosure of which is incorporated herein in its entirety by this reference. To ensure that transmural ablation is achieved, some practitioners have been utilizing larger needles (e.g., 18 g needles, which have a needle diameter of 1.27 mm), which generally form a larger lesion than a smaller needle under otherwise similar conditions. Such larger needles also form larger punctures in a subject's skin and create similarly larger trauma regions as the needle is inserted into the subject to position the needle tip at the tissue to be ablated, which may increase patient discomfort, increase the procedure time due to difficulties of inserting such larger needles, and prolong the time needed for recovery and otherwise increase harmful side effects of treatment.

### DISCLOSURE OF THE INVENTION

Disclosed are needle assemblies for high frequency ablation that include a needle comprising an electrically conductive portion and a bore extending at least partially along a length of the needle. At least one conductive member extends at least partially through the bore and a portion of the at least one conductive member is physically and electrically connected to the electrically conductive portion of the needle.

In some embodiments, described are ablation systems including a needle assembly as described herein, a high frequency probe electrode adapted for at least partial insertion into the bore of the needle and electrical communication with the at least one conductive member, and a high frequency current source configured for electrical connection to the high frequency probe electrode.

In additional embodiments, described are methods of forming needle assemblies for use in ablation procedures include disposing at least one conductive member within a needle and physically and electrically connecting the at least one conductive member to an electrically conductive portion of the needle.

In still other embodiments, described are methods of high frequency ablation include directing current at high frequency to a high frequency probe electrode disposed in a bore of a needle and flowing the current from the high frequency probe electrode, through at least one conductive member disposed within the bore of the needle and contacting the high frequency probe electrode, to a portion of the at least one conductive member that is physically and electrically connected to the needle.

### BRIEF DESCRIPTION OF THE DRAWINGS

FIG. 1 is a side view of a needle assembly for use in ablation procedures in accordance with an embodiment hereof.

FIG. 2 is a partial cross-sectional view of the needle assembly of FIG. 1.

FIG. 3 is an enlarged cross-sectional view of a distal end of the needle assembly of FIG. 1.

FIG. 4 is an enlarged cross-sectional view of a proximal end of the needle assembly of FIG. 1.

FIG. 5 is a partial cross-sectional side view of the needle assembly of FIG. 1 including an electrode.

FIG. 6 is an enlarged cross-sectional view of the distal end of the needle assembly of FIG. 5.

FIG. 7 is a side view of a needle assembly for use in ablation procedures in accordance with another embodiment hereof.

FIG. 8 is a partial cross-sectional view of the needle assembly of FIG. 7.

FIG. 9 is an enlarged cross-sectional view of a distal end of the needle assembly of FIG. 7.

FIG. 10 is an enlarged cross-sectional view of a proximal end of the needle assembly of FIG. 7.

FIG. 11 is a partial cross-sectional side view of the needle assembly of FIG. 7 including an electrode.

FIG. 12 is an enlarged cross-sectional view of the distal end of the needle assembly of FIG. 11.

FIG. 13 is a simplified cross-sectional view of a needle assembly for use in ablation procedures during use.

### MODE(S) FOR CARRYING OUT THE INVENTION

The illustrations presented herein are not meant to be actual views of any particular needle assembly or component thereof, but are merely idealized representations that are employed to describe illustrative embodiments. Thus, the drawings are not necessarily to scale and relative dimensions may have been exaggerated or understated for the sake of clarity. Additionally, elements common between figures may retain the same or similar numerical designation.

Disclosed is a needle assembly for use in ablation procedures (e.g., high frequency ablation) that reduces impedance of the needle assembly. In particular, embodiments of needle assemblies for use in ablation procedures include a conductive member that increases contact between an electrically conductive distal end the needle and an electrode inserted into a bore of the needle. Such embodiments may act to reduce the impedance of the needle assemblies and more readily transmit a signal (e.g., a complete RF frequency) to the needle tip.

As used herein, the terms “distal” and “proximal” are terms of convenience for describing relative relationships and refer to an orientation of a needle assembly with respect to the health care provider when in use. For example, a distal end or portion of a needle assembly is the portion of the needle closest to a subject and furthest from a practitioner during use of the needle assembly and a proximal end or portion of the needle assembly is the portion of the needle closest to the practitioner and furthest from the subject during use of the needle assembly.

As used herein, the term “high frequency” with respect to alternating electrical current means and includes electrical currents alternating at frequencies sufficiently high to cause lesions to form in human or animal tissue. High frequency alternating currents include, for example, currents alternating at radio frequencies (e.g., frequencies between about 3 kHz and 300 GHz) and currents alternating at microwave frequencies (e.g., frequencies between about 300 MHz and 300 GHz).

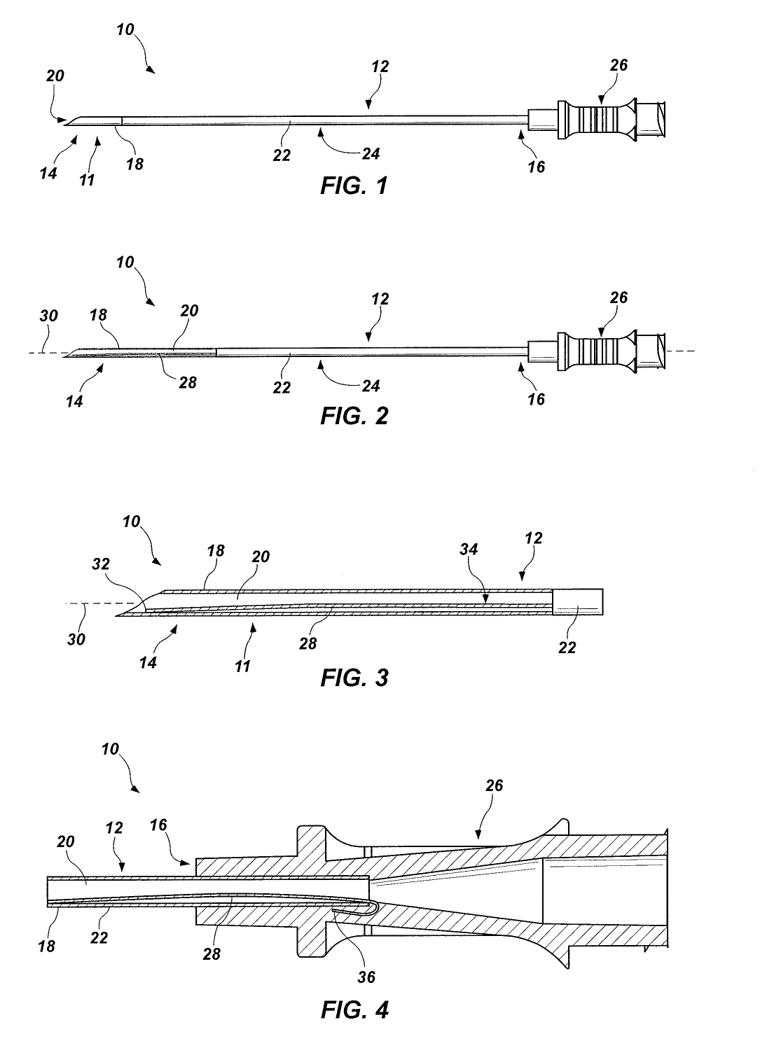

Referring to FIG. 1, a side view of a needle assembly 10 for use in ablation procedures is shown. The needle assembly 10 includes a needle 12 having an electrically conductive portion 11 and an electrically insulated portion 24. For example, the needle 12 includes an elongated hollow member 18 (e.g., a cannula) configured for at least partial insertion into a subject and a dielectric material 22 on the exterior surface of the elongated hollow member 18 in some embodiments. The elongated hollow member 18 may be formed from or associated with an electrically conductive material suitable for use in medical applications, such as, for example, medical grade stainless steel, titanium, copper, or alloys thereof. The elongated hollow member 18 defines a bore 20 extending at least partially along a length of the needle 12 between a distal end 14 and a proximal end 16 of the needle 12, through which a fluid (e.g., a medicament, an analgesic, a solution, a biological administration) may be delivered and into which an electrode (e.g., a high frequency probe electrode) may be inserted. The elongated hollow member 18 may have a circular cross-section, and the bore 20 may have a correspondingly circular cross-section in some embodiments. In other embodiments, the elongated hollow member 18 may have a non-circular cross-section, such as, for example, oval, rectangular, polygonal, or irregular, and the bore 20 may, but need not, have a correspondingly non-circular cross-section (not shown). In still other embodiments, the bore 20 may have a cross-sectional shape different from a cross-sectional shape of the elongated hollow member 18.

As noted previously, the needle 12 may include a dielectric material 22 disposed on or associated with an exterior surface of the elongated hollow member 18 in some embodiments. The dielectric material 22 may be formed from an electrically insulating material suitable for use in medical applications (e.g., acrylonitrile butadiene styrene (ABS)). The dielectric material 22 covers the elongated hollow member 18 at the proximal end 16 of the needle 12 and at the intermediate portion 24 of the needle 12. The electrically conductive material of the elongated hollow member 18 is exposed (i.e., not covered by the dielectric material 22) at the distal end 14 of the needle 12.

The bore 20 defined by a surface of the elongated hollow member 18 may also be at least partially exposed (i.e., not covered by the dielectric material 22). Contact or other electrical connection between a current-carrying member (e.g., a probe electrode) and the surfaces of the elongated hollow member 18 defining the bore 20 may enable the current to be conducted from the current-carrying member, through the elongated hollow member 18, to the distal end 14 of the needle 12. In this way, the distal end 14 of the needle 12 may be configured to ablate tissue in contact with or proximate to the distal end 14 of the needle 12 utilizing ablation, while the intermediate portion 24 and the proximal end 16 of the needle 12 may be configured to prevent or impede the flow of current to tissue in contact with or proximate to the intermediate portion 24 and the proximal end 16 of the needle 12.

In other embodiments, the needle 12 may comprise an elongated dielectric member (e.g., a tube formed from dielectric material) connected to a conductive distal end (e.g., a tip formed from conductive material connected to the tube) where the conductive distal end is in electrical communication with a current-carrying member.

The proximal end 16 of the needle 12 may be connected to a needle hub 26. The needle hub 26 is typically configured to remain outside a subject during an ablation procedure. The needle hub 26 may be configured for handling by a practitioner, such as, for example, by including a portion curved to accommodate a grip, by including ribs or other gripping members to facilitate manipulation of the needle assembly 10, by being formed from an insulative material, or combinations thereof. The needle hub 26 may also be configured for connection to another structure or device, such as, for example, by including a Luer-Lok® connection, a Luer-Slip connection, or a threaded connection. The needle hub 26 may be configured to enable other structures, devices, or substances to pass through the needle hub 26 into the bore 20 of the needle 12.

Referring to FIG. 2, a partial cross-sectional view of the needle assembly 10 of FIG. 1 is shown. At least one conductive member 28 is electrically connected to a portion of the needle 12. For example, the conductive member 28 is coupled to the electrically conductive portion 11 of the needle 12 at a location proximate the distal end 14 (e.g., at or near a tip or terminal portion of the needle 12). At least a portion of the conductive member 28 may be formed of an electrically conductive material suitable for use in medical applications, such as, for example, medical grade stainless steel, titanium, copper, or alloys thereof. As specific, non-limiting examples, the conductive member 28 may be formed from a medical grade stainless steel (e.g., 302V or 304V type stainless steel). The conductive member 28 may have any cross-sectional shape, such as, for example, circular, oval, rectangular, etc., and may comprise, for example, a ribbon, a wire, a cord, a strand, a plurality of ribbons, a plurality of wires, a plurality of cords, a plurality of strands, or combinations thereof at least partially formed of electrically conductive material. As shown in FIG. 2, the conductive member 28 may comprise a single ribbon extending through at least a portion of the bore 20 of the needle 12 in some embodiments. The conductive member 28 reduces the cross-sectional area of at least a portion of the bore 20 formed in the needle 12 in which another structure or device can be disposed (see, e.g., FIG. 3). In some embodiments, the conductive member 28 extends along at least substantially the entire length of the needle 12, from proximate the distal end 14, through the intermediate portion 24, to proximate the proximal end 16 in some embodiments. For example, in a 10 cm needle, the conductive member 28 extends along the entire length of the needle 12 or a length slightly less than the entire length of the needle 12 (e.g., a length slightly less than 10 cm such 9.9 cm or less). In other embodiments, the conductive member 28 may extend along only a portion or portions of the length of the needle 12. For example, in a 10 cm needle, the conductive member 28 extends along a length less than the entire length of the needle 12 (e.g., 9 cm, 8 cm, 7 cm, 6 cm, 5 cm or less).

The needle 12 is at least substantially straight along its entire length in some embodiments. For example, a central axis 30 of the bore 20 defined by the elongated hollow member 18 may be at least substantially linear. More specifically, the central axis 30 of the bore 20 defined by the elongated hollow member 18 may deviate from a straight line by less than 3 mm, less than 2 mm, or even less than 1 mm. In other embodiments, the needle 12 may be curved along all or a portion of its length.

Referring to FIG. 3, an enlarged cross-sectional view of the distal end 14 of the needle 12 of FIG. 1 is shown. A distal end 32 of the conductive member 28 may be in electrical communication with (e.g., physically and electrically connected to) the electrically conductive distal end 14 of the needle 12. For example, the distal end 32 of the conductive member 28 may be, e.g., soldered, welded, brazed, or adhered utilizing conductive epoxy to an interior surface of the elongated hollow member 18 defining the bore 20 at the distal end 14 of the needle 12. As another example, the distal end 32 of the conductive member 28 may be, e.g., embedded within the conductive material of the distal end 14 during formation of the distal end 14. Current (e.g., high frequency, alternating current) flowing from the conductive member 28 to the conductive material of the distal end 14 of the needle 12 concentrates at the distal end 14 of the needle 12 because of the fixed, direct electrical connection between the conductive member 28 and the distal end 14.

An intermediate portion 34 of the conductive member 28 is free-floating within the bore 20 of the needle 12 in some embodiments (e.g., the intermediate portion 35 of the conductive member 28 may extend along the bore 20 proximate the central axis 30 of the needle 10). For example, the conductive member 28 may not be directly physically attached to the elongated hollow member 18, with the exception of the distal end 32 of the conductive member 28, and may freely move within the bore 20 in some embodiments. In some embodiments, the intermediate portion 34 of the conductive member 28 may be intermittently or even continuously electrically connected to the elongated hollow member 18 of the needle 12, depending on how it is positioned within the bore 20, due to electrical communication between (e.g., via contact with or proximity to) the intermediate portion 34 of the conductive member 28 and the interior surface of the elongated hollow member 18 of the needle 12. In other embodiments, the intermediate portion 34 of the conductive member 28 may be intermittently or continuously fixedly attached to the elongated hollow member 18 of the needle 12 or to another device or structure disposed in the bore 20 defined by the elongated hollow member 18 of the needle 12.

The distal end 14 of the needle 12 is pointed in some embodiments. For example, the distal end 14 may comprise a pointed tip defined by a bevel surface extending across the central axis 30 of the needle 12 at an oblique angle (see, e.g., FIG. 3). As a specific, non-limiting example, the distal end 14 of the needle 12 may be configured as a Tuohy needle, which generally includes a slight curve at the distal end 14, or other conventional needle tip configurations, such as, for example, Hustead needles, Weiss needles, and Eldor needles. In other embodiments, the distal end of the needle 12 is blunt or otherwise not pointed. The distal end 14 of the needle 12 is also open such that the bore 20 is in communication with the environment axially outward from the distal end 14 of the needle 12 in some embodiments. In other words, the bevel surface of the pointed distal end 14 may surround the bore 20 to define an opening at the distal end 14, and the central axis 30 of the needle 12 may pass through the opening without intersecting material of the elongated hollow member 18. In this way, a fluid (e.g., a medicament, an analgesic, a solution, or a biological administration) may be delivered through the bore 20 to tissue at the distal end 14 of the needle 12 via the opening.

In other embodiments, the distal end 14 of the needle 12 may not be open, but fluids may still be delivered utilizing, for example, side port openings 44 formed in the needle 12 as shown and described with reference to FIG. 9 below.

Referring to FIG. 4, an enlarged cross-sectional view of the proximal end 16 of the needle 12 of FIG. 1 is shown. The proximal end 16 of the needle 12 is secured to the needle hub 26. A proximal end 36 of the conductive member 28 is likewise secured to the needle hub 26 in some embodiments. For example, the conductive member 28 may be bent to curve around the proximal end 16 of the needle 12 such that the proximal end 36 of the conductive member 28 is located outside the bore 20. The proximal end 36 may be embedded in the material of the needle hub 26. In this way, the distal end 32 (FIG. 3) and the proximal end 36 of the conductive member 28 may be fixed while the intermediate portion 34 of the conductive member 34 is free-floating within the bore 20 of the needle 12. In other embodiments, the proximal end 36 may be secured to the inner surface of the elongated hollow member 18 defining the bore 20. In still other embodiments, the proximal end 36 may be free-floating.

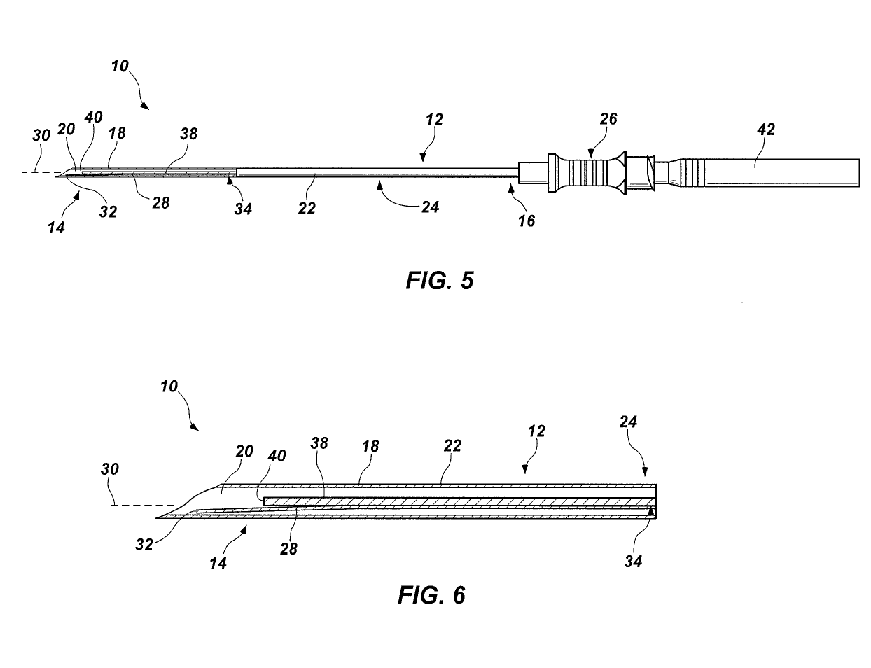

Referring to FIG. 5, a partial cross-sectional side view of the needle assembly 10 of FIG. 1 including an electrode 38 (e.g., a high frequency probe electrode) is shown. The electrode 38 is configured to connect to a current source (e.g., an electrical radio frequency (RF) current generator) and provide current (e.g., high frequency, alternating current) to the distal end 14 of the needle 12 to ablate tissue. The electrode 38 is inserted into the bore 20 of the needle 12 and makes contact with the conductive member 28. The electrode 38 may comprise, for example, an RF probe thermocouple. Such an RF probe thermocouple may comprise, for example, an outer portion of conductive material and a core wire extending within the outer portion. The optional thermocouple portion of the electrode 38 may be disposed at a distal end 40 of the electrode 38. Suitable RF probe electrodes and other high frequency probe electrodes are available, for example, from Epimed International, Inc., the New York Plant of which is located at 141 Sal Landrio Dr., Johnstown, N.Y., 12095. The electrode 38 may be inserted into the bore 20 of the needle 12 through the needle hub 26. An electrode hub 42 may engage with the needle hub 26 when the electrode 38 is fully inserted into the needle 12 to secure the electrode 38 in place. Such an electrode hub 42 may connect to a current source, such as, for example, an electrical RF current generator or an electrical microwave frequency current generator. Suitable current sources are available, for example, from Stryker Instruments of 4100 Milham Ave., Kalamazoo, Mich., 49001.

Referring to FIG. 6, an enlarged cross-sectional view of the distal end 14 of the needle 12 of FIG. 5 is shown. The distal end 40 of the electrode 38 may be disposed within the bore 20 at the distal end 14 of the needle 12 when the electrode 38 is fully inserted into the needle 12. In this way, the optional thermocouple portion of the electrode 38 may provide feedback about the temperature at the distal end 14 of the needle 12, which is configured to ablate tissue.

When the electrode 38 is disposed within the bore 20, the electrode 38 may be in electrical communication with (e.g., via contact with or proximity to) one or more of the conductive member 28 and the inner surface of the elongated hollow member 18 of the needle 12 defining the bore 20. In such an embodiment, the current flowing through the electrode 38 is enabled to pass from the electrode 38 to the conductive member 28, the elongated hollow member 18 of the needle 12, or both. The current flows from one or more of the electrode 38 and the conductive member 28 (i.e., from the electrode 38 via the conductive member 28) particularly to the distal end 14 of the needle 12, enabling the distal end 14 of the needle 12 to ablate tissue.

In some embodiments, one or more of the needle 12 and the electrode 38 may be disposable. For example, the needle 12 and the electrode 38 may be discrete, separately formed components that are connected to one another to form the needle assembly 10. After an ablation procedure is performed, the electrode 38 may be withdrawn from the bore 20 of the needle 12, cleaned, and subsequently reused with another needle to ablate tissue. The needle 12 is discarded in such embodiments. In other embodiments, the needle 12 may be cleaned and subsequently reused with another electrode 38, the needle 12 and the electrode 38 may be cleaned and subsequently be reused with another electrode and another needle, respectively, or the needle 12 and the electrode 38 may be cleaned and subsequently reused with one another. In still other embodiments, the needle 12 and the electrode 38 may be permanently assembled to one another, for example, by permanently affixing the electrode hub 42 to the needle hub 26 or by establishing permanent electrical contact between the electrode 38 and the elongated hollow member 18, the conductive member 28, or both.

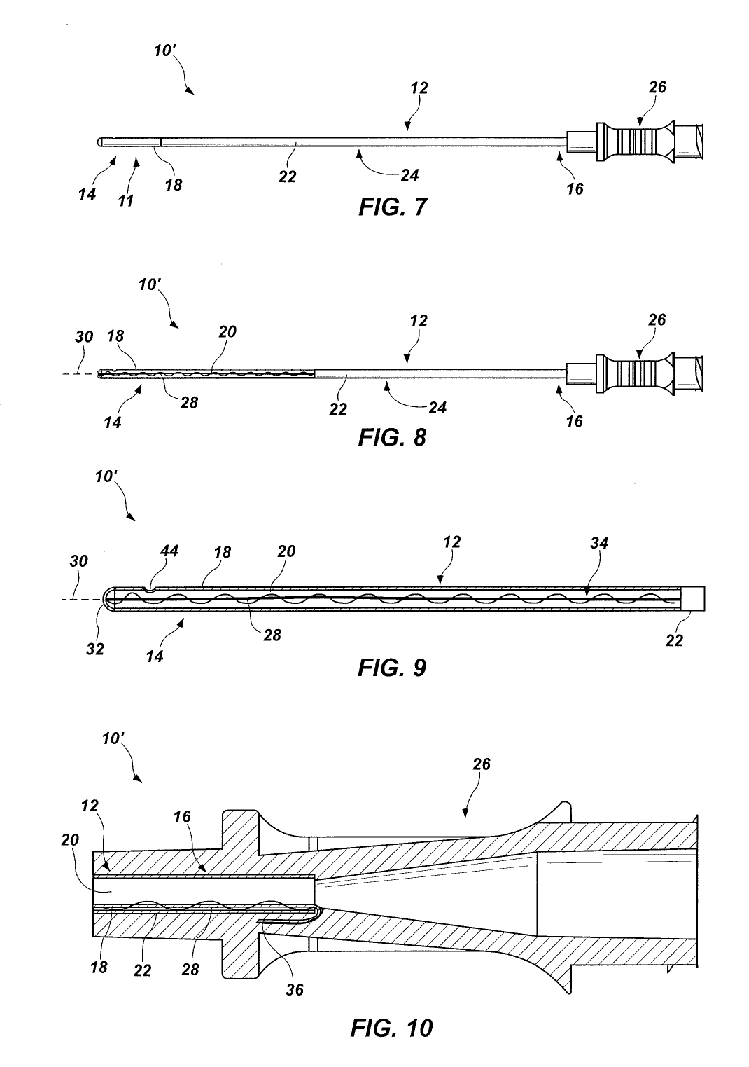

Referring to FIG. 7, a side view of another embodiment of a needle assembly 10′ for use in ablation procedures is shown. The needle assembly 10′ and its associated components may be similar to the needle assembly 10 discussed above in relation to FIGS. 1 through 6 and includes a needle 12 having an electrically conductive portion 11 and an electrically insulated proximal portion 24. For example, the needle 12 comprises an elongated hollow member 18 (e.g., a cannula) configured for at least partial insertion into a subject, and a dielectric material 22 on the exterior surface of the elongated hollow member 18 in some embodiments.

Referring to FIG. 8, a partial cross-sectional view of the needle assembly 10′ of FIG. 7 is shown. One or more conductive members 28 (e.g., a plurality) are physically and electrically connected to the electrically conductive distal end 14 of the needle 12 in some embodiments. The conductive members 28 are formed of an electrically conductive material suitable for use in medical applications, such as, for example, medical grade stainless steel, titanium, copper, or alloys thereof. The conductive members 28 may comprise, for example, ribbons, wires, cords, or strands at least partially formed from electrically conductive material. The conductive members 28 reduce the cross-sectional area of the bore 20 in which another structure or device can be disposed. The conductive members 28 extend along at least substantially the entire length of the needle 12, from the distal end 14, through the intermediate portion 24, to the proximal end 16, in some embodiments. In other embodiments, the conductive members 28 may extend along only a portion or portions of the length of the needle 12.

Referring to FIG. 9, an enlarged cross-sectional view of the distal end 14 of the needle 12 of FIG. 7 is shown. Distal ends 32 of the conductive members 28 are physically and electrically connected to the distal end 14 of the needle 12. More specifically, the distal ends 32 of the conductive members 28 may be, e.g., soldered, welded, brazed, or adhered utilizing conductive epoxy to an interior surface of the elongated hollow member 18 defining the bore 20 at the distal end 14 of the needle 12. As another example, the distal ends 32 of the conductive members 28 may be, e.g., embedded within the conductive material of the distal end 14 during formation of the distal end 14.

Intermediate portions 34 of the conductive members 28 are free-floating within the bore 20 of the needle 12 in some embodiments. For example, the conductive members 28 may not be directly physically attached to the elongated hollow member 18, with the exception of the distal ends 32 of the conductive members 28, and may freely move within the bore 20. In some embodiments, the intermediate portions 34 of the conductive members 28 may be intermittently or even continuously electrically connected to the elongated hollow member 18 of the needle 12 because of physical contact between the intermediate portions 34 of the conductive members 28 and the interior surface of the elongated hollow member 18 of the needle 12.

The distal end 14 of the needle 12 of FIG. 7 is blunt or otherwise not pointed in some embodiments. More specifically, the distal end 14 of the needle may comprise a hemispherical cap such that the bore does not open axially to an exterior of the needle 12 in such embodiments. In other words, the central axis 30 may intersect the body of the elongated hollow member 18 at the hemispherical cap located at the distal end 14 of the needle 12. In some embodiments, one or more side port openings 44 provides communication between the exterior of the needle 12 and the bore 20 of the needle 12 such that a fluid (e.g., a medicament, an analgesic, a solution, or a biological administration) is deliverable to the exterior of the needle 12 proximate the distal end 14 through the side port opening(s) 44. In still other embodiments, the bore 20 of the needle 12 may not directly communicate with the exterior of the needle 12, and fluids may not be deliverable through the bore 20 of the needle 12.

Referring to FIG. 10, an enlarged cross-sectional view of the proximal end 16 of the needle 12 of FIG. 7 is shown. The proximal end 16 of the needle 12 is secured to the needle hub 26. A proximal end 36 of the conductive member 28 is likewise be secured to the needle hub 26 in some embodiments. More specifically, the conductive member 28 is bent to curve around the proximal end 16 of the needle such that the proximal end 36 of the conductive member 28 is located outside the bore 20. The proximal end 36 is embedded in the material of the needle hub 26. In this way, the distal end 32 (FIG. 9) and the proximal end 36 of the conductive member 28 are fixed while the intermediate portion 34 of the conductive member 34 is free-floating within the bore 20 of the needle 12. In other embodiments, the proximal end 36 may be secured to the inner surface of the elongated hollow member 18 defining the bore 20. In still other embodiments, the proximal end 36 may be free-floating.

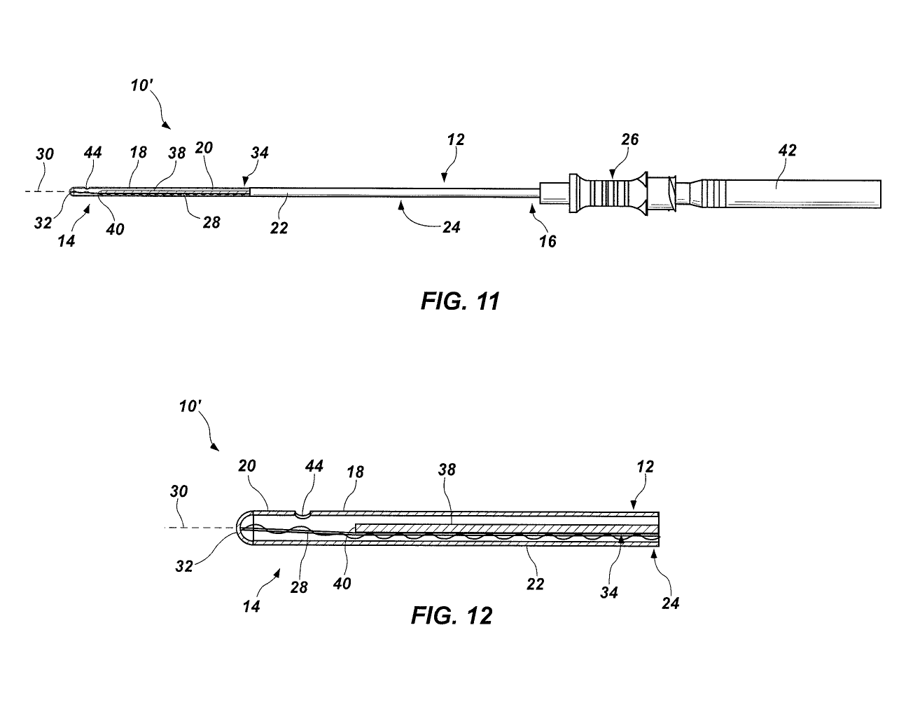

Referring to FIG. 11, a partial cross-sectional side view of the needle assembly 10′ of FIG. 7 including an electrode 38 is shown. The electrode 38 is configured to connect to a current source and provide current to the distal end 14 of the needle 12 to ablate tissue. The electrode 38 is inserted into the bore 20 of the needle 12 and physically and electrically contacts the conductive member 28. The electrode 38 may comprise, for example, an RF probe thermocouple. The electrode 38 may be inserted into the bore 20 of the needle 12 through the needle hub 26. An electrode hub 42 may engage with the needle hub 26 when the electrode 38 is fully inserted into the needle 12. Such an electrode hub 42 may connect to a current source, such as, for example, an electrical RF current generator or an electrical microwave frequency current generator.

Referring to FIG. 12, an enlarged cross-sectional view of the distal end 14 of the needle 12 of FIG. 11 is shown. A distal end 40 of the electrode 38 is disposed within the bore 20 at the distal end 14 of the needle 12 when the electrode 38 is fully inserted into the needle 12. In this way, an optional thermocouple portion of the electrode 38 may provide feedback about the temperature at the distal end 14 of the needle 12, which is configured to ablate tissue.

When the electrode 38 is disposed within the bore, a portion of the electrode 38 is in electrical communication with the conductive member 28, the inner surface of the elongated hollow member 18 of the needle 12 defining the bore 20, or both. Accordingly, current flowing through the electrode 38 passes (e.g., directly passes) to the conductive member 28, the elongated hollow member 18 the needle 12, or both. The current flows particularly to the distal end 14 of the needle 12, enabling the needle 12 to ablate tissue.

When forming a needle assembly (e.g., the needles assemblies 10, 10′ described above), the proximal end 36 of the conductive member 28 optionally may be bent to form a hook or crook shape. The conductive member 28 may be inserted into the bore 20 of the needle 12, and the optional hook may engage with the proximal end 16 of the needle 12 to retain the proximal end 36 of the conductive member 28 outside the bore 20 and to ensure proper positioning of the distal end 32 of the conductive member 28 with respect to the distal end 14 of the needle 12. The distal end 32 of the conductive member 28 is in electrical communication with the electrically conductive distal end 14 of the needle 12. For example, the distal end 32 may be soldered, welded, brazed, or adhered utilizing conductive epoxy to an inner surface of the elongated hollow member 18 defining the bore 20 at the distal end 14 of the needle 12. As another, non-limiting example, the distal end 32 of the conductive member 28 may be embedded within the electrically conductive material of the distal end 14 of the needle 12 during formation of the distal end 14. The proximal end 36 of the conductive member 28 may optionally be fixed as well. For example, the proximal end 36 of the conductive member 28 optionally may be embedded within the needle hub 26 by forming the needle hub 26 around the proximal end 16 of the needle 12, for example, by injection molding. As another example, the proximal end 36 of the conductive member 28 may optionally be connected to the elongated hollow member 18 at the proximal end 16. An electrode 38 is optionally inserted into the bore 20 and is in electrical communication with the conductive member 28. By inserting the electrode 38 into the bore 20 along with the conductive member 28, the area of physical and electrical contact between the electrode 38 and other electrically conductive components of the needle assembly 10, 10′, such as, for example, the elongated hollow member 18 of the needle 12 and the conductive member 28, is increased relative to a needle assembly lacking such a conductive member 28.

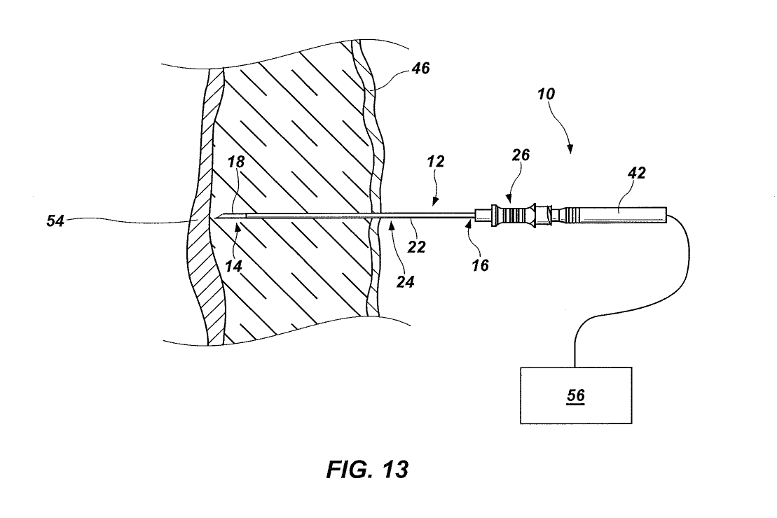

Referring to FIG. 13, a simplified cross-sectional view of a needle assembly 10 for use in ablation procedures is shown during use. The needle 12 may puncture the skin 46 of a subject and the distal end 14 of the needle 12 may be positioned proximate to a neural structure of the subject to be ablated (e.g., adjacent nervous tissue 54). In some embodiments, the distal end 14 of the needle 12 may physically contact the nervous tissue 54 to be ablated. In other embodiments, the needle 12 may puncture the skin 46 of a subject in other regions and the distal end 14 of the needle 12 may be positioned adjacent tissue to be ablated that is located elsewhere within the subject and may be nervous tissue or tissue of another type. A fluid may optionally be administered to the subject via the needle 12, such as, for example, to dull or numb pain receptors in the ablation area. Current is directed to the electrode 38 (FIGS. 5, 6, 11, and 12). For example, the electrode hub 42 may be electrically connected to a current source 56, and current may flow from the current source to the electrode 38. The current may alternate at one or more radio frequencies in some embodiments. The current flows from the electrode 38, through conductive components of the needle 12 (e.g., a conductive member 28 (FIGS. 2 through 6 and 8 through 12) in electrical communication with the distal end 14 of the needle 12 or the conductive member 28 and conductive material of the elongated hollow member 18), to the distal end 14 of the needle 12. The current flows from the distal end 14 of the needle 12 into the nervous tissue 54 or other tissue to be ablated. The high concentration of the current at the distal end 14 of the needle 12 ablates the nervous tissue.

As the current dissipates through adjoining tissues to a grounding pad, typically a large surface area grounding pad located at or near the leg of a subject, the high frequency alternating current ceases to ablate tissue because of its reduced concentration. The needle assembly 10 may ablate a larger lesion in the nervous tissue 54 or other tissue to be ablated than a similar needle assembly lacking the conductive member 28 (FIGS. 2 through 6 and 8 through 12) under otherwise similar circumstances because of the increased electrical contact area between the electrode 38 (FIGS. 5, 6, 11, and 12) and other electrically conductive components of the needle assembly 10. In this way, a smaller gauge needle 12 may be used to ablate tissue that previously may have required utilizing a larger gauge needle to achieve complete or transmural ablation of the nervous tissue 54 or other tissue to be ablated.

### EXAMPLES

Two needle assemblies were provided for experimentation, one needle assembly including a conductive member physically and electrically connected to the electrically conductive distal end thereof and the other needle assembly lacking such a conductive member. The needles for both assemblies were 20 g (i.e., 0.981 mm diameter) straight needles. The conductive member of the one needle assembly was a wire formed from 304V medical grade stainless steel. The distal ends of the needles of both needle assemblies were inserted into the same cut of chicken. The cut of chicken was held at temperatures between about 20° C. and about 25.5° C. A high frequency, alternating current source was set to maintain an 80° C. ablation temperature for a 90-second ablation time. After the ablation time expired, the resulting lesions in the cuts of chicken were measured. Specifically, the major and minor axes of the generally oval-shaped lesions were measured utilizing calipers. The cross-sectional area of each lesion was then calculated utilizing the formula:

Area of Burn=π*(Major Axis Length/2)*(Minor Axis Length/2).

This procedure was repeated for 50 trials.

The average cross-sectional area of the lesions formed by the needle assembly including the conductive member was 0.113297 in² (73.09 mm²). By contrast, the average cross-sectional area of the lesions formed by the needle assembly lacking such a conductive member was 0.099901 in² (64.45 mm²). Thus, the needle assembly including the conductive member formed a lesion 0.013396 in² (8.64 mm²) larger than the lesion formed by the needle assembly lacking such a conductive member, on average. This was unexpected.

Embodiments of needle assemblies described above may be particular useful in ablation procedures as the presence of the conductive member may enhance electrical communication between the electrode and the needle by increasing one or more of the number and physical and electrical contacts between the electrode and the needle as compared to a needle assembly lacking the conductive member. For example, the physical and electrical contact area between the electrode and the conductive material of the elongated hollow member, in embodiments where the elongated hollow member comprises a conductive material, is greater because the space within at least a portion the bore is reduced by the conductive member. In addition, the total physical and electrical contact area between electrically conductive components of the needle and the electrode is increased because the conductive member establishes physical and electrical contacts not previously made utilizing needle assemblies lacking such conductive members. The increased physical and electrical contact between electrically conductive components may reduce the impedance of the needle assembly and more readily transmit a complete signal (e.g., a complete RF frequency) to the needle tip, which enables the needle assembly to ablate a larger quantity of tissue than a similar needle assembly lacking the conductive member under otherwise similar conditions. In addition, it is believed that the increased physical contact between electrically conductive components may reduce degradation of the current electrical signal as it flows from the electrode to the distal end of the needle as compared to a similar needle assembly lacking the conductive member under otherwise similar conditions.

In such embodiments, lesions formed by flowing current through the conductive member to the distal end of the needle may be formed more quickly and may be larger than lesions formed by needles lacking such a conductive member in otherwise similar conditions (e.g., starting temperature, current frequency and amplitude, duration of procedure, etc.). Accordingly, the conductive member may enable health care professionals to utilize smaller gauge needles while still enabling complete (i.e., transmural) ablation of tissue to be removed.

While the present disclosure has been described herein with respect to certain example embodiments, those of ordinary skill in the art will recognize and appreciate that it is not so limited. Rather, many additions, deletions, and modifications to the embodiments described herein may be made without departing from the scope of the disclosure, embodiments of which are hereinafter claimed, including legal equivalents. In addition, features from one disclosed embodiment may be combined with features of another disclosed embodiment while still being encompassed within the scope of the disclosure as contemplated by the inventor.

1. A needle assembly for use in an ablation procedure, the needle assembly comprising:
a needle comprising an electrically conductive portion and a bore extending at least partially along a length of the needle; and
at least one conductive member extending at least partially through the bore, a portion of the at least one conductive member being physically and electrically connected to the electrically conductive portion of the needle.

2. The needle assembly of claim 1, further comprising an electrode at least partially disposed within the bore of the needle and in electrical communication with the at least one conductive member.

3. The needle assembly of claim 2, wherein the at least one conductive member is positioned within the bore in physical contact with the electrode.

4. The needle assembly of claim 3, wherein the at least one conductive member is physically and electrically connected to a distal end of the needle.

5. The needle assembly of claim 4, wherein the at least one conductive member is physically and electrically connected to the needle at a location proximate to an opening formed at the distal end of the needle.

6. The needle assembly of claim 1, wherein a proximal end of the at least one conductive member is embedded within a needle hub connected to the proximal end of the needle.

7. The needle assembly of claim 6, wherein a distal end and the proximal end of the at least one conductive member are fixed, and an intermediate portion of the at least one conductive member is free-floating within the bore of the needle.

8. The needle assembly of claim 1, wherein the at least one conductive member comprises at least one of a flat ribbon, a wire, a cord, a plurality of flat ribbons, a plurality of wires, and a plurality of cords.

9. The needle assembly of claim 1, wherein the needle comprises an elongated hollow member of electrically conductive material defining the bore and a dielectric material disposed on a portion of an exterior surface of the elongated hollow member at the proximal end and along an intermediate portion of the needle, and wherein another portion of the exterior surface of the elongated hollow member is exposed at the distal end of the needle.

10. The needle assembly of claim 9, wherein a central axis of the bore defined by the elongated hollow member is at least substantially linear.

11. The needle assembly of claim 1, wherein the distal end of the needle comprises a pointed end.

12. The needle assembly of claim 1, wherein the at least one conductive member comprises a plurality of conductive members.

13. The needle assembly of claim 1, together with
a high frequency probe electrode adapted for at least partial insertion into the bore of the needle of the needle assembly and in electrical communication with the at least one conductive member; and
a high frequency current source configured for electrical connection to the high frequency probe electrode.

14. The needle assembly of claim 13, wherein the high frequency probe electrode comprises an RF probe thermocouple having a thermocouple disposed at the distal end of the needle.

15. The needle assembly of claim 13, wherein the high frequency current source is configured to flow alternating current at radio frequency to the high frequency probe electrode.

16. The needle assembly of claim 13, wherein the at least one conductive member is positioned within the bore to be in physical contact with the high frequency probe electrode when the high frequency probe is at least partially inserted in the bore of the needle.

17. A method of making the needle assembly of claim 1, the method comprising:
disposing at least one conductive member within the bore of the needle; and
physically and electrically connecting the at least one conductive member to an electrically conductive portion of the needle.

18. The method according to claim 17, further comprising securing a proximal end of the at least one conductive member within a needle hub connected to a proximal end of the needle.

19. The method according to claim 18, further comprising extending a portion of the at least one conductive member freely through the bore of the needle.

20. The method according to claim 18, further comprising physically and electrically connecting the at least one conductive member to at least another electrically conductive portion of the needle.

21. The method according to claim 18, further comprising:
inserting a high frequency probe electrode into the bore of the needle; and
contacting the high frequency probe electrode with the at least one conductive member.

22. A method of high frequency ablation utilizing the needle assembly of claim 1, the method comprising:
directing current at high frequency to a high frequency probe electrode disposed in the bore of the needle; and
flowing the current from the high frequency probe electrode, through the at least one conductive member disposed within the bore of the needle and contacting the high frequency probe electrode, to a portion of the at least one conductive member that is physically and electrically connected to the needle.
